(19) United States
(12) Patent Application Publication
(10) Pub. No.: US 2011/0263009 A1
(43) Pub. Date: Oct. 27, 2011

(54) METHOD FOR THE FILTRATION OF A BIOREACTOR LIQUID FROM A BIOREACTOR; CROSS-FLOW MEMBRANE MODULE, AND BIOREACTOR MEMBRANE SYSTEM

(75) Inventors: Sjoerd Hubertus Jozef Vellinga, Tjalleberd (NL); Jelle Faber, Jubbega (NL); Harm Van Dalfsen, Steenwijk (NL)

(73) Assignees: Paques I.P. B.V.; Paques Bio Systems B.V.

(21) Appl. No.: 13/126,678

(22) PCT Filed: Oct. 30, 2009
(86) PCT No.: PCT/NL2009/050658

§ 371 Date: Jul. 14, 2011

(30) Foreign Application Priority Data
Oct. 30, 2008 (EP) 08167972.2

(51) Int. Cl. C12M 1/12; B01D 63/00; B01D 63/06; B01D 61/00

(52) U.S. Cl. 435/297.1; 210/650; 210/321.72

(57) ABSTRACT

The invention provides a method for the filtration of a bioreactor liquid (10) from a bioreactor (1) with a cross-flow membrane module (20) comprising one or more membranes (40). The method comprises feeding part of the bioreactor liquid (10) to a liquid inlet (21) of the cross-flow membrane module (20), transporting the bioreactor liquid (10) through the cross-flow membrane module (20) in a cross-flow mode, and removing a retentate (12) from a liquid outlet (22) of the cross-flow membrane module (20). The cross-flow membrane module (20) is arranged to allow a liquid downward flow of the bioreactor liquid (10) through the cross-flow membrane module (20). The method further comprises providing the liquid downward flow and a downward gas flow of a gas (30) through the cross-flow membrane module (20). Fouling of the membranes is relatively good prevented or reduced in time. Further, the liquid flow may be reduced relative to conventional configurations.

### FIELD OF THE INVENTION

The invention relates to a method for the filtration of a bioreactor liquid from a bioreactor, a cross-flow membrane module and a bioreactor membrane system.

### BACKGROUND OF THE INVENTION

The use of membranes in water cleaning/purification is well known in the art. US2004007527, for instance, describes that an element for use in ultra filtration or micro filtration of potable water has a large number of small diameter hollow fibre membranes attached between two headers. Side plates attached to the sides of the headers define vertical flow channels containing the membranes. The elements may be placed side by side and stacked on top of each other to form cassettes having continuous vertical flow channels through the entire cassette. The membrane modules or cassettes may be arranged to cover a substantial part of the cross sectional area of an open tank. Tank water may flow upwards or downwards through the flow channels. A tank may be deconcentrated by at least partially emptying and refilling the tank with fresh water while permeation continues. Excess tank water created during deconcentration may flow generally upwards through the modules and out through a retentate outlet or overflow at the top of the tank.

WO03095371 describes a method and system for the treatment of water by flocculation carried out in one or several flocculation steps and by separating the flocculi in a downstream sedimentation stage and by reducing the floccular slurry. The water flows in the sedimentation stage along membrane filter plates which are oriented towards each other and in relation to the inflow of the water to be treated in such a way that cross-flow filtration, dead-end filtration and sedimentation occurs to a desired extent.

Further, U.S. Pat. No. 4,964,987 describes a cross flow filter apparatus and method using an open tank having a first liquid retaining section, a second filter retaining section and a third solids collecting section in fluid communication with each other. A filter assembly is retained within the second section and includes a filter panel having a generally vertically disposed filter membrane surface, preferably with submicron pores. Filtrate is removed by applying low vacuum pressure, in the range of about 5 inches vacuum pressure (Mercury), in communication with the filter panel such that filtrate is drawn through the pores of the filter membrane surface at a flow rate Qₒᵤₜ. Fluid to be filtered is cross flowing vertically downward across the filter membrane surface at a flow rate Q_{X}, such that the horizontal velocity Vₕ of fluid drawn through the filter membrane surface is less than the vertical velocity Vᵥ of the cross flowing unfiltered fluid. The excess high velocity cross flowing unfiltered fluid imparts a shearing action to particles resting on the filter membrane surface to rehabilitate the filter membrane, thereby continuously offering a clean filter membrane surface for continued filtration. Excess unfiltered cross flowing fluid is recirculated between the first and second sections of the tank, while allowing entrained particles to settle to the solids collecting section of the tank as the recirculated fluid mixes with incoming unfiltered fluid prior to recirculation and discharge vertically downward across the filter membrane surfaces.

WO2006/058902 describes a filtering system for water and waste water, which comprises at least one container in which aerated filter modules are disposed. At least one feed compartment is provided for jointly feeding suspension to be filtered to the filter modules. The inventive system is characterized by a feed distribution compartment through which the suspension to be filtered is introduced into the feed compartment, the feed distribution compartment being partially guided around the feed compartment. The invention allows to reduce the space required below the filter modules for feeding the suspension.

US 2004/0007527 describes an element for use in ultrafiltration or microfiltration of potable water which has a large number of small diameter hollow fibre membranes attached between two headers. Side plates attached to the sides of the headers define vertical flow channels containing the membranes. The elements may be placed side by side and stacked on top of each other to form cassettes having continuous vertical flow channels through the entire cassette. The membrane modules or cassettes may be arranged to cover a substantial part of the cross sectional area of an open tank. Tank water may flow upwards or downwards through the flow channels. A tank may be deconcentrated by at least partially emptying and refilling the tank with fresh water while permeation continues. Excess tank water created during deconcentration may flow generally upwards through the modules and out through a retentate outlet or overflow at the top of the tank

WO2008/048594 describes a method of assisting the removal of phosphorous from wastewater in a wastewater treatment system comprising a membrane bioreactor having at least one membrane, the method comprising forming a mixture of a gas and liquid medium; adding a coagulant to the mixture; applying the mixture and added coagulant to a surface of the membrane and filtering a permeate through a wall of the membrane.

### SUMMARY OF THE INVENTION

Tubular membranes are mainly used in the process industries for ultra filtration usages. One of the disadvantages of these systems operated in the cross flow mode is the high consumption of (water) recycle pump energies especially when they are used in the bio membrane systems.

To overcome this problem and to keep the membrane tubes clean the membrane systems may be mounted vertically and the flow through the module is in upward direction while air is injected on the bottom part. This air injection has preferably to create so much turbulence that the flow over the module can be reduced and still keep the tubes clean. The problem of such system may however be that the distribution of water and air may not evenly be divided over all membrane tubes. Some tubes may function as riser and some as downer and some may not have any flow. The tubes that function as riser may attract all the water and air and create a high velocity, which sucks water from the top of the module via downers. In the tubes which function as downer the flow velocity may be rather low and the shear will be too low for the tube to be kept clean. Here, the term “tube” may refer to “tubular membranes” or “membrane tubes”.

However, advantageously, it appears that when downwards aeration is combined with a downwards flow, a substantially even distribution of gas (such as air) over all tubes of the module may be achieved. Such configuration may further be substantially self adjusting: when the downwards flow is increased in a tube, more air may be sucked into a tube, the specific gravity of the air water mixture decreases, the resistance will increase and the flow will decrease so that less air is sucked into the tube. This is exactly the opposite of upward aeration, which is destabilising: when one of the tubes is filled with water, the velocity in the pipe will increase and it will suck in air till the velocity decreases again.

Hence, it is an aspect of the invention to provide an alternative method for the filtration of a bioreactor liquid from a bioreactor, which preferably further obviates one or more of above-described drawbacks. It is further an aspect to provide an alternative cross-flow membrane module, which preferably further obviates one or more of above-described drawbacks. It is yet further an aspect to provide an alternative bioreactor membrane system, which preferably further obviates one or more of above-described drawbacks.

According to a first aspect, the invention provides a method for the filtration of a bioreactor liquid from a bioreactor with a cross-flow membrane module comprising one or more membranes, wherein the method comprises:

a. feeding part of the bioreactor liquid to a liquid inlet of the cross-flow membrane module,

b. transporting the bioreactor liquid through the cross-flow membrane module in a cross-flow mode, and

c. removing a retentate from a liquid outlet of the cross-flow membrane module,

wherein the cross-flow membrane module is arranged to allow a liquid downward flow (QL) of the bioreactor liquid through the cross-flow membrane module, and wherein the method further comprises providing the liquid downward flow (QL) of the bioreactor liquid through the cross-flow membrane module and a downward gas flow (QG) of a gas through the cross-flow membrane module.

Advantageously, fouling of the membranes is also relatively good prevented or reduced in time. Further, the liquid flow and/or gas flow, especially the superficial liquid flow (see below), may be reduced relative to conventional configurations. Hence, in this way less liquid may be circulated. Hence, the bioreactor liquid is transported through the cross-flow membrane module in a liquid downward flow.

In a specific embodiment, the invention provides a method wherein the gas holdup in the liquid downward flow (QL) through the cross-flow membrane module is in the range of about 0.5-25 vol. %, especially about 5-25 vol. % of the liquid downward flow (QL in m³/h). Under these circumstances, good anti fouling may be obtained, while minimizing energy consumption.

In an embodiment, the superficial liquid flow velocity is in the range of 0.1-2.5, especially 0.2-1.5 m/s. Here, the superficial liquid flow velocity is given in m/s, and is the flow over the membrane (cross-flow). In prior art applications, this may be substantially higher, such as for instance about 3-4 m/s or more. Hence, the liquid downward flow may have a superficial liquid flow velocity (over the cross-flow membrane(s) of the cross-flow membrane module) in the range of 0.2-1.5 m/s.

Gas may be injected in the cross-flow membrane module in different ways. The gas may be separately injected, but may also be injected in the bioreactor liquid before flowing over the membranes. In a specific embodiment, the bioreactor liquid and the gas are mixed before flowing through the cross-flow membrane module in a cross-flow mode.

In general, at least part of the bioreactor liquid will be circulated through the cross-flow membrane module. Hence, in a specific embodiment, a retentate of the cross-flow membrane module is fed to the bioreactor. Herein, the term “retentate” refers to the part of a solution in a filtration process that does not cross the membrane. “Permeate” is liquid that has passed the pores of the membrane. In general, permeate can also be indicated as “purified water” or “clean water” or “filtrated water”.

Especially preferred are tubular membranes, through which the bioreactor liquid may be transported. Such membranes may for instance be ultra filtration membranes or micro filtration membranes. Such membranes are commercially available. Hence, in a specific embodiment, the invention provides a method wherein the one or more membranes comprise one or more tubular membranes, wherein the one or more tubular membranes are arranged to allow the liquid downward flow (QL) of the bioreactor liquid and the downward gas flow (QG) of the gas through the one or more tubular membranes in a cross-flow mode. In an embodiment, the membrane comprises an ultra filtration membrane.

The gas may comprise for instance one or more of air, nitrogen, natural gas and biogas. Biogas may for instance be obtained from the bioreactor (comprising the bioreactor liquid).

According to a further aspect, the invention provides a cross-flow membrane module comprising one or more membranes, a liquid inlet for a bioreactor liquid and a liquid outlet for a retentate of the cross-flow membrane module, wherein the cross-flow membrane module further comprises a gas inlet for a gas, and wherein the cross-flow membrane module, the liquid inlet, the liquid outlet, and the gas inlet are arranged to allow a liquid downward flow (QL) of the bioreactor liquid and a downward gas flow (QG) of the gas through the cross-flow membrane module in a cross-flow mode. Such cross-flow membrane module may especially be used to perform the above described method of the invention.

According to yet a further aspect, the invention also provides a membrane bioreactor system comprising a bioreactor and the cross-flow membrane module as described herein, arranged external from the bioreactor, wherein the bioreactor is arranged to comprise a bioreactor liquid, and wherein the bioreactor is in liquid communication with the liquid inlet of the cross-flow membrane module.

As mentioned above, at least part of the bioreactor liquid may circulate from the bioreactor to cross-flow membrane module and back. Hence, preferably, the bioreactor is in liquid communication with the liquid outlet of the cross-flow membrane module, and the membrane bioreactor system is arranged to circulate at least part of the bioreactor liquid through the cross-flow membrane module.

Therefore, the invention advantageously provides further the use of a liquid downward flow (QL) of a bioreactor liquid and a downward gas flow (QG) of a gas through a cross-flow membrane module (such as described herein), comprising one or more membranes, in a cross-flow mode for filtrating the bioreactor liquid. Such liquid downward flow (QL) of a bioreactor liquid and a downward gas flow (QG) of a gas through the cross-flow membrane module, comprising one or more membranes, in a cross-flow mode for filtrating the bioreactor liquid, may in addition advantageously be used for reducing fouling of the one or more membranes.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIG. 1 schematically depicts an embodiment of a membrane bioreactor system according to an embodiment of the invention; and

FIGS. 2a/2b schematically depict in more detail an embodiment of the cross-flow membrane module of an embodiment of the invention.

### DESCRIPTION OF PREFERRED EMBODIMENTS

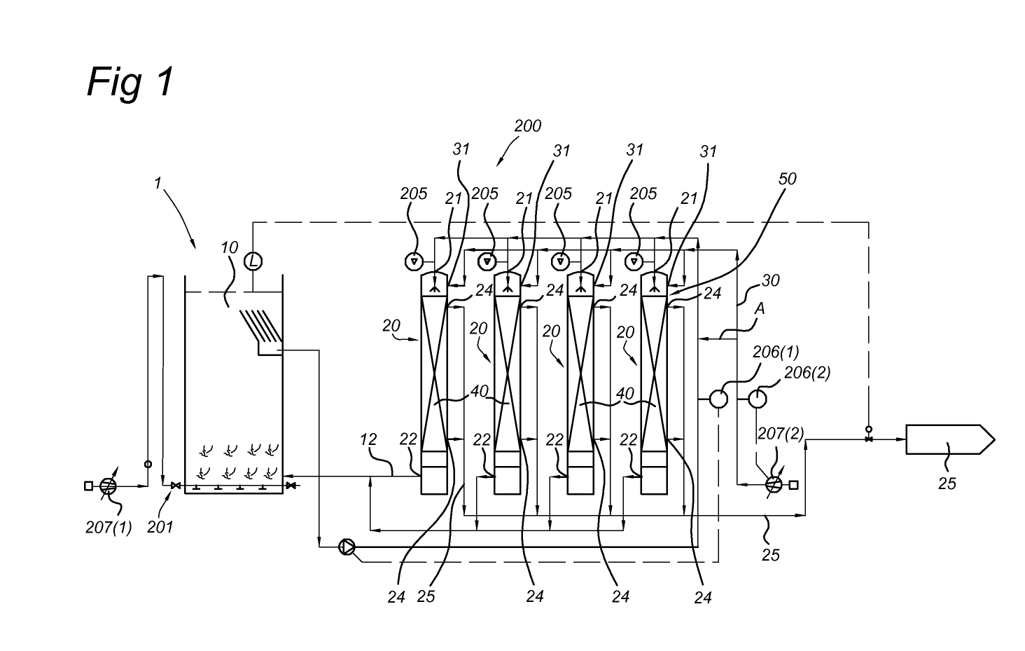

FIG. 1 schematically depicts a membrane bioreactor system, indicated with reference 200 comprising a bioreactor 1 and a cross-flow membrane module 20. The cross-flow membrane module 20 is arranged external from the bioreactor 1. The bioreactor 1 is arranged to comprise bioreactor liquid 10. The bioreactor 1 is in liquid communication with a liquid inlet 21 of the cross-flow membrane module 20. In this way, bioreactor liquid 10 can be transported to the cross-flow membrane module 20.

Bioreactor liquid may be transported to one or more cross-flow membrane modules 20. In this example, 4 of such modules 20 are schematically depicted, each having an inlet 21. The membrane modules 20 comprise membranes 40.

The cross-flow membrane module 20 may comprise one or more membranes 40, a liquid inlet 21 for the bioreactor liquid 10 and a liquid outlet 22 for a retentate 12 of the cross-flow membrane module 20 (i.e. bioreactor liquid that has not passed through the membrane). The cross-flow membrane module 20 further comprises a gas inlet 31 for a gas 30. The cross-flow membrane module 20, the liquid inlet 21, the liquid outlet 22, and the gas inlet 31 are arranged to allow a liquid downward flow of the bioreactor liquid 10 and a downward gas flow of the gas 30 through the cross-flow membrane module 20 in a cross-flow mode. As is shown in FIG. 1, the gas 30 and liquid 10 are provided to the cross-flow membrane module 20 at the top, and retentate 12 is extracted from the bottom of the cross-flow membrane module 20 and leaves the cross-flow membrane module 20 via outlet 22. Thus, in a liquid downward flow, liquid 10 is transported through the cross-flow membrane module 20. The gas holdup in the liquid flow in the liquid 10 through the cross-flow membrane module 20 may for instance be in the range of 0.5-25 vol. %, such as 5-25 vol. %, or more especially 10-25 vol. %. Thus, the method of the invention further comprises providing the liquid downward flow of the bioreactor liquid 10 and a downward gas flow of a gas 30 through the cross-flow membrane module 20.

Permeate, indicated with reference 25, may be extracted from the membrane (permeate side, not shown in the figure), and may leave the cross-flow membrane module via outlet(s) 24. Optionally, part of it may be rerouted back to the bioreactor 1; and part, of all may be used for other applications. Reference L refers to an optional level meter.

The liquid outlet 22 may be in liquid contact with the bioreactor 1. In this way, the membrane bioreactor system 200 may be arranged to circulate at least part of the bioreactor liquid 10 through the cross-flow membrane module 20. Bioreactor liquid 10 may so pass several times the cross-flow membrane module 20.

The liquid 10 in the bioreactor 10 may be aerated by means of an aeration system 201, receiving air (or another gas or gas mixture) from for instance a compressor 207(1).

The liquid flow, and thus the downward flow, of the bioreactor liquid 10 may be controlled by means of pressure sensors 205 and a flow meter 206(1); the gas flow, i.e. the downward gas flow, may be controlled by a gas flow meter 206(2). Gas may be provided by another compressor 207(2) (right hand side in the schematic drawing). Optionally, gas 30 may be mixed with the liquid 10 before entering the cross-flow membrane module 20, or at least before coming into contact with the membrane 40 within the cross-flow membrane module 20. An embodiment thereof is schematically indicated with a dashed line with reference A.

In this way, bioreactor liquid 10 from a bioreactor 1 can be filtrated with a cross-flow membrane module 20 by feeding part of the bioreactor liquid 10 to the liquid inlet 21 of the cross-flow membrane module 20, transporting the bioreactor liquid 10 through the cross-flow membrane module 20 in a cross-flow mode, and removing a retentate 12 from a liquid outlet 22 of the cross-flow membrane module 20, in such a way that a liquid downward flow of the bioreactor liquid 10 through the cross-flow membrane module 20 is allowed, and a downward gas flow of the gas 30 through the cross-flow membrane module 20 is obtained. The bioreactor liquid 10 and the gas 30 can be mixed before flowing through the cross-flow membrane module 20 in a cross-flow mode (as shown in an embodiment by flow A. Further, retentate 12 of the cross-flow membrane module 20 can fed to the bioreactor 1. Cleaner bioreactor liquid, or “purified” bioreactor liquid, or “filtered bioreactor liquid”, in general water, indicated with reference 25, can leave the cross-flow membrane module 20 via one or more openings 24.

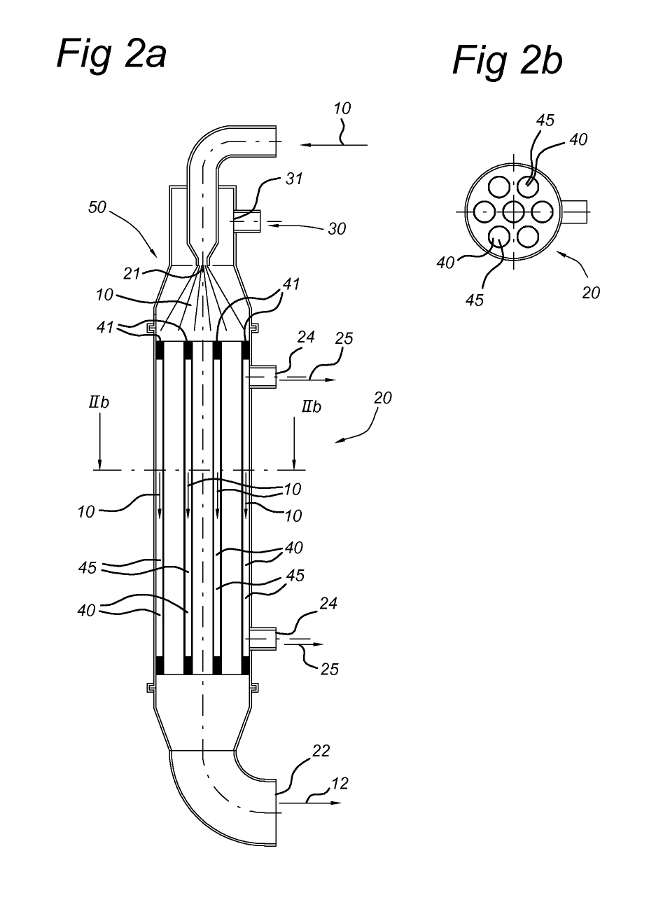

An embodiment of the cross-flow membrane module 20 is depicted in more detail in FIGS. 2a and 2b. Bioreactor liquid 10 enters cross-flow membrane module 20 via inlet 21 and gas 30 via inlet 30. The liquid 10 comprising gas bubbles flows along the membranes 40, here tubular membranes 45, which may for instance be ultra filtration membranes or micro filtration membranes. Retentate 12 may escape from the cross-flow membrane module 20 via outlet(s) 22. Purified liquid 25 (permeate) leaves the membranes via membrane openings at the permeate side of the membranes 40 and can escape from the reactor via outlet(s) 24.

The membrane module 20 according to an embodiment of the invention may thus comprise a module head 50, arranged to provide liquid 10 and gas 30 to the membrane(s), especially tubular membrane(s), at the top(s), indicated with reference 41, of the membrane(s), thereby allowing a downward flow of the liquid 10. Such module head 50 may comprise the liquid inlet 20 and the gas inlet 30.

Whereas in conventional systems the superficial liquid flow may for instance be about 3-4 m/s, the superficial liquid flow in the invention may for instance be as low as about 0.5 m/s. Hence, the invention may provide an energy reduction of the pump providing the superficial liquid flow of up to 60-70%, while having a good or even better foul reduction, relative to methods wherein the membrane modules are arranged to allow a liquid upward flow of the bioreactor liquid 10 through the cross-flow membrane module.

The term “substantially” herein, such as in “substantially all emission” or in “substantially consists”, will be understood by the person skilled in the art. The term “substantially” may also include embodiments with “entirely”, “completely”, “all”, etc.

Hence, in embodiments the adjective substantially may also be removed. Where applicable, the term “substantially” may also relate to 90% or higher, such as 95% or higher, especially 99% or higher, even more especially 99.5% or higher, including 100%. The term “comprise” includes also embodiments wherein the term “comprises” means “consists of”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The apparatus herein are amongst others described during operation. As will be clear to the person skilled in the art, the invention is not limited to methods of operation or apparatus in operation.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A method for the filtration of a bioreactor liquid from a bioreactor with a cross-flow membrane module (20) comprising one or more membranes, wherein the method comprises:
a. feeding the bioreactor liquid or part thereof to a liquid inlet of the cross-flow membrane module,
b. transporting the bioreactor liquid through the cross-flow membrane module in a cross-flow mode, and
c. removing a retentate from a liquid outlet of the cross-flow membrane module,
wherein the cross-flow membrane module is arranged to allow a liquid downward flow of the bioreactor liquid through the cross-flow membrane module, and wherein the method further comprises providing said liquid downward flow of the bioreactor liquid and a downward gas flow of a gas through the cross-flow membrane module.

2. The method according to claim 1, wherein the gas holdup in the liquid downward flow through the cross-flow membrane module is in the range of 5-25 vol. %.

3. The method according to claim 1, wherein the liquid downward flow has a superficial liquid flow velocity in the range of 0.2-1.5 m/s.

4. The method according to claim 1, wherein the bioreactor liquid and the gas are mixed before flowing through the cross-flow membrane module in a cross-flow mode.

5. The method according to claim 1, wherein a retentate of the cross-flow membrane module is fed to the bioreactor

6. The method according to claim 1, wherein the one or more membranes comprise one or more tubular membranes, arranged to allow the liquid downward flow of the bioreactor liquid and the downward gas flow of the gas through the one or more tubular membranes in a cross-flow mode.

7. The method according to claim 1, wherein the gas comprises one or more of air and biogas.

8. A cross-flow membrane module comprising one or more membranes, a liquid inlet for a bioreactor liquid and a liquid outlet for a retentate of the cross-flow membrane module, wherein the cross-flow membrane module further comprises a gas inlet for a gas, and wherein the cross-flow membrane module, the liquid inlet, the liquid outlet, and the gas inlet are arranged to allow a liquid downward flow of the bioreactor liquid and a downward gas flow of the gas through the cross-flow membrane module in a cross-flow mode.

9. A membrane bioreactor system comprising a bioreactor and a cross-flow membrane module according to claim 8, arranged external from the bioreactor, wherein the bioreactor is arranged to comprise a bioreactor liquid, and wherein the bioreactor is in liquid communication with the liquid inlet of the cross-flow membrane module.

10. The membrane bioreactor system, wherein the bioreactor is in liquid communication with the liquid outlet of the cross-flow membrane module, and wherein the membrane bioreactor system is arranged to circulate at least part of the bioreactor liquid through the cross-flow membrane module.

11-12. (canceled)
